(19) United States
(12) Patent Application Publication
(10) Pub. No.: US 2022/0211298 A1
(43) Pub. Date: Jul. 7, 2022

(54) SMART BACK POSTURE CORRECTOR

(71) Applicant: XIAOWEI Chen, Shenzhen (CN)

(72) Inventor: XIAOWEI Chen, Shenzhen (CN)

(21) Appl. No.: 17/702,795

(22) Filed: Mar. 24, 2022

(30) Foreign Application Priority Data
Sep. 16, 2021 (CN) 202111084929.3

(51) Int. Cl. A61B 5/11; A61B 5/00

(52) U.S. Cl. CPC A61B 5/1116; A63B 23/0244; A61B 5/6823

(57) ABSTRACT

The present disclosure relates to the technical field of a posture corrector, and in particular to a smart back posture corrector, which comprises a machine body for attaching to a user's back and a casing, wherein the machine body and an adhesive sheet for attaching the machine body to the user's back are provided in the casing; a control circuit board, a gyroscope and a physical action part are provided in the machine body, when an angular motion data monitored by the gyroscope exceeds a set value, the control circuit board controls the physical action part to turn on, and the physical action part outputs physical actions acting on the user's back.

### TECHNICAL FIELD

The present disclosure relates to the technical field of a posture corrector, and in particular to a smart back posture corrector.

### BACKGROUND

Hunchback is a relatively common spinal deformity, which is a morphological change caused by the posterior protrusion of the thoracic spine, and it is more common in elderly spinal deformity, incorrect sitting posture, rickets, and other diseases such as ankylosing spondylitis.

At present, for most people, especially students and children, due to incorrect sitting posture, the spine including the cervical spine and the lumbar spine is in an incorrect bending state, in the long term, it is easy to cause the spine deformation, thus resulting in hunchback.

In the prior art, physical equipment such as straps or collars are usually used to correct the sitting posture, so as to ensure correct sitting posture and avoid hunchback. However, the physical equipment cannot accurately determine whether the sitting posture is correct, and with the passage of use time, the physical equipment is easily deformed and cannot prevent hunchback.

### SUMMARY

An objective of embodiments of the present disclosure is to provide a smart back posture corrector, which can remind the user and judge the sitting posture of the user accurately.

One embodiment of the present disclosure provides a smart back posture corrector, comprising a machine body for attaching to a user's back and a casing, wherein the machine body and an adhesive sheet for attaching the machine body to the user's back are provided in the casing; a control circuit board, a gyroscope and a physical action part are provided in the machine body, when an angular motion data monitored by the gyroscope exceeds a set value, the control circuit board controls the physical action part to turn on, and the physical action part outputs physical actions acting on the user's back.

Preferably, a circuit of the control circuit board comprises a control chip U1, a power supply chip U2, a first operational amplifier U3, a second operational amplifier U4, a first capacitor C1, a second capacitor C2, a third capacitor C3, a fourth capacitor C4, a fifth capacitor C5, a sixth capacitor C6, a first resistor R1, a second resistor R2, a third resistor R3, a fourth resistor R4, a fifth resistor R5, a sixth resistor R6, a seventh resistor R7, an eighth resistor R8, a ninth resistor R9 and an external power supply VCC; an X-axis output port Xout, a Y-axis output port Yout and a Z-axis output port Zout of the control chip U1 are grounded through the first capacitor C1, the second capacitor C2 and the third capacitor C3 respectively, a sleep control port Sleep of the control chip U1 is connected to an operating voltage VDD through the first resistor R1, a mode selection port g_Select and a self test function port Self Test of the control chip U1 are directly grounded.

Preferably, when the angular motion data exceeds the set value, the X-axis output port Xout, the Y-axis output port Yout and the Z-axis output port Zout of the control chip U1 output voltages in three directions of the X-axis, Y-axis and Z-axis respectively, in such a way that the physical action part is turned on.

Preferably, the control circuit board comprises a wireless communication component for communicating with an external mobile terminal, and the mobile terminal is used for controlling the working state of the smart back posture corrector.

Preferably, the physical action part is a vibrator.

Preferably, the physical action part is a pulser.

Preferably, a cavity for placing the machine body is provided in the casing, an external interface communicating with the cavity is formed on the casing, and a charging port is provided on the machine body, when the machine body is placed in the cavity, the charging port and the external interface are aligned and connected with each other.

Preferably, the casing comprises a bottom casing and an upper casing that swings up and down relative to the bottom casing, a side of the upper casing is hinged with a side of the bottom casing, and the upper casing and the bottom casing are enclosed to form the cavity.

Preferably, a bottom frame is provided in the cavity, the bottom frame is arranged on the bottom casing, a bottom space for accommodating the adhesive sheet is provided between the bottom frame and the bottom casing, and the bottom frame is provided with a lower recess for placing the machine body.

Preferably, the bottom frame comprises a frame plate, an upper of the frame plate forms the lower recess, a bottom of the frame plate extends downward with a bottom brace, the bottom brace is connected to the bottom casing by magnetic attraction, and the bottom space is formed between the frame plate and the bottom casing.

Preferably, the upper casing has an inner surface facing the bottom casing, a positioning plate is connected to the inner surface of the upper casing, the positioning plate is provided with a plurality of positioning holes, and when the upper casing is covered on the bottom casing, an upper of the machine body is penetrated between a plurality of positioning holes.

Preferably, a side wall of each positioning hole extends toward the inner surface of the upper casing to form a positioning ring piece, and the positioning ring piece abuts against an outer circumference of the machine body.

Preferably, an alcohol cotton is provided in the casing.

Compared with the prior art, the smart back posture corrector provided by the embodiments of the present disclosure has the following beneficial effects: the machine body is attached to the user's back, when the user's sitting posture is incorrect, such as skewed or bent, the gyroscope will perform angular motion, and when the angular motion data exceeds the set value, the physical action part will perform physical actions to remind the user that the sitting posture is incorrect, until the user adjusts the sitting posture, in such a way that the smart back posture corrector provided by the present embodiment can accurately remind the user and judge the sitting posture of the user, so as to prevent hunchback caused by incorrect sitting posture, and the angular motion data and a data of the physical action part will be transmitted to the mobile terminal for data statistics.

### BRIEF DESCRIPTION OF THE DRAWINGS

In order to more clearly illustrate the technical solutions in the embodiments of the present disclosure, drawings used in the description of the embodiments will be briefly described below, obviously, the drawings in the following description are some embodiments of the present disclosure, those of ordinary skill in the art can also obtain other drawings based on these drawings without any creative efforts.

FIG. 1 is a three-dimensional explosion diagram of a smart back posture corrector provided by the embodiment of the present disclosure.

FIG. 2 is a local three-dimensional explosion diagram of the smart back posture corrector provided by the embodiment of the present disclosure.

FIG. 3 is a circuit diagram of the control circuit board of the smart back posture corrector provided by the embodiment of the present disclosure.

### DESCRIPTION OF THE EMBODIMENTS

To make the objective, technical solutions and advantages of the present disclosure clearer and more comprehensible, the present disclosure will be further described in detail below with reference to the drawings and the embodiments. It should be understood that the specific embodiments described herein are only used to explain the disclosure and are not used to limit the disclosure.

The implementation of the present disclosure will be described in detail in combination with specific embodiments.

It is to be noted that when an element is described as “fixed on” or “provided on” another element, it may be directly or indirectly on the another element. When one element is described as “connected to” another element, it may be directly or indirectly connected to the another element.

The same or similar reference signs in the drawings of the present embodiment correspond to the same or similar components; in the description of the present disclosure, it should be understood that if there is an orientation or positional relationship indicated by terms “upper”, “lower”, “left”, “right”, etc., the orientation or positional relationship is based on the orientation or positional relationship shown in the drawings, and is only for the convenience of describing the present disclosure and simplifying the description, rather than indicating or implying that the indicated device or element must have a specific orientation or be constructed and operated in a specific orientation. Therefore, the terms describing the positional relationship shown in the drawings are only for exemplary description, and cannot be understood as a limitation of the present disclosure. For those of ordinary skill in the art, the specific meaning of the above terms can be understood according to the specific circumstances.

In addition, terms “first” and “second” are merely for the purpose of description, but cannot be understood as the indication or implication of relative importance or as the implicit indication of the number of the designated technical features. Therefore, features defined by “first” and “second” may explicitly or implicitly include one or more such features. In the description of the present disclosure, unless otherwise clearly and specific stated, “a plurality of” means two or more than two.

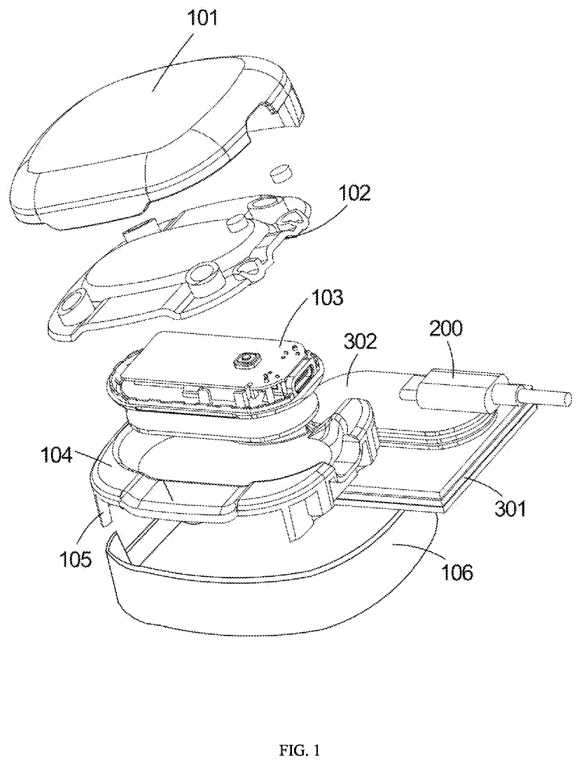

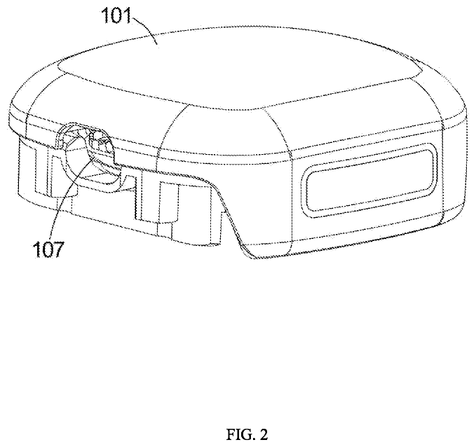

Referring to FIG. 1 and FIG. 2, a preferred embodiment of the present disclosure is provided.

A smart back posture corrector, comprising a machine body 103 and a casing, wherein the machine body 103 is used for attaching to a user's back, a cavity for placing the machine body 103 is provided in the casing, the cavity is provided with an adhesive sheet 302, a back surface of the machine body 103 is provided with an attachment area where the adhesive sheet 302 is attached. When the machine body 103 is needed for use, remove the machine body 103 from the cavity of the casing, attach one side of the adhesive sheet 302 to the attachment area of the machine body 103, and then attach the other side of the adhesive sheet 302 to the user's back. When the machine body 103 is not needed, remove the machine body 103 from the user's back and the adhesive sheet 302 attached to the machine body 103, or the adhesive sheet 302 can keep being attached to the machine body 103, and the machine body 103 is accommodated in the cavity of the casing.

The machine body 103 is provided with a control circuit board, a gyroscope, and a physical action part, the control circuit board serves as a control center, which controls the operation of the machine body 103. The control circuit board has a wireless communication component that communicate with an external mobile terminal, the mobile terminal controls the working state of the smart back posture corrector by communicating with the control circuit board, in such a way that parameters of the machine body 103 can be set or the working state of the machine body 103 can be controlled through the APP (application program) in the mobile terminal; when an angular motion data monitored by the gyroscope exceeds a set value, the control circuit board controls the physical action part to turn on, and the physical action part outputs physical actions acting on the user's back.

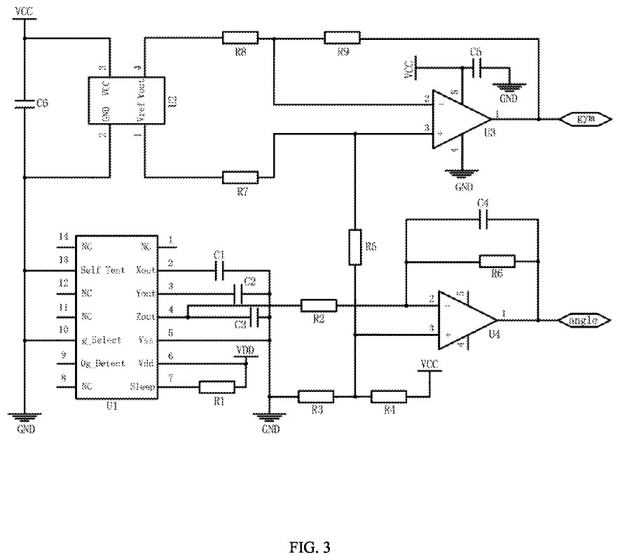

Referring to FIG. 3, FIG. 3 is a circuit diagram of the control circuit board of the smart back posture corrector provided by the embodiment of the present disclosure. A circuit of the control circuit board comprises a control chip U1, a power supply chip U2, the first operational amplifier U3, the second operational amplifier U4, the first capacitor C1, the second capacitor C2, the third capacitor C3, the fourth capacitor C4, the fifth capacitor C5, the sixth capacitor C6, the first resistor R1, the second resistor R2, the third resistor R3, the fourth resistor R4, the fifth resistor R5, the sixth resistor R6, the seventh resistor R7, the eighth resistor R8, the ninth resistor R9 and an external power supply VCC.

In the present embodiment, the control chip U1 is a MMA7361 chip. The second pin (X-axis output port Xout), the third pin (Y-axis output port Yout) and the fourth pin (Z-axis output port Zout) of the control chip U1 are grounded through the first capacitor C1, the second capacitor C2 and the third capacitor C3 respectively. The fifth pin (ground port Vss) of the control chip U1 is directly grounded, the sixth pin (power port Vdd) of the control chip U1 is directly connected to an operating voltage VDD, the operating voltage VDD is the voltage when the control chip U1 operates normally, which can be obtained by converting the voltage value of the external power supply VCC to the voltage value of the control chip U1 operating normally through a step-down LM1117 chip or a circuit composed of discrete devices such as resistors and capacitors. The seventh pin (sleep control port Sleep) of the control chip U1 is connected to the operating voltage VDD through the first resistor R1, the tenth pin (mode selection port g_Select) and the thirteenth pin (self test function port Self Test) of the control chip U1 are directly grounded, and other pins of the control chip U1 are suspended (not connected).

The MMA7361 chip is an acceleration sensor, which has a preset control program, when the angular motion data exceeds the set value, the X-axis output port Xout, the Y-axis output port Yout and the Z-axis output port Zout of the control chip U1 are controlled to output the voltage in three directions of the X-axis, Y-axis and Z-axis respectively, in such a way that the physical action part is turned on.

The power supply chip U2 provides a reference voltage Vref for the first operational amplifier U3 and the second operational amplifier U4 to compare the input voltage. The first pin (reference voltage input port Vref) of the power supply chip U2 is connected to the third pin (in-phase input port) of the first operational amplifier U3 through the seventh resistor R7, the second pin (ground port GND) of the power supply chip U2 is directly grounded, the third pin (power port VCC) of the power supply chip U2 is directly connected to the external power supply VCC, and the fourth pin (voltage output port Vout) of the power supply chip U2 is connected to the second pin (inverse-phase input port) of the first operational amplifier U3 through the eighth resistor R8, and the sixth capacitor C6 is connected between the second pin and the third pin of the power supply chip U2.

The first pin (output port) of the first operational amplifier U3 is connected to a physical action part driving circuit, which is used to drive the physical action part to output the physical actions acting on the user's back. To explain the disclosure briefly, only the circuit of the control circuit board is shown in FIG. 3, the specific circuit composition of the physical action part driving circuit is not shown, and a port named gym is used to represent the physical action part driving circuit. The ninth resistor R9 is connected between the first pin and the second pin of the first operational amplifier U3, the fourth pin (ground port) of the first operational amplifier U3 is directly grounded, the fifth pin (power port) of the first operational amplifier U3 is directly connected to the external power supply VCC, and the fifth pin of the first operational amplifier U3 is grounded through the fifth capacitor C5.

The first pin (output port) of the second operational amplifier U4 is connected to a gyroscope acquisition circuit, which is used to acquire the angular motion data of the machine body 103. To explain the disclosure briefly, only the circuit of the control circuit board is shown in FIG. 3, the specific circuit composition of the gyroscope acquisition circuit is not shown, and a port named angle is used to represent the gyroscope acquisition circuit. The second pin (inverse-phase input port) of the second operational amplifier U4 is connected to the fourth pin of the control chip U1 through the second resistor R2, the third pin (in-phase input port) of the second operational amplifier U4 is grounded through the third resistor R3, the third pin of the second operational amplifier U4 is connected to the external power supply VCC through the fourth resistor R4, the third pin of the second operational amplifier U4 is connected to the first pin of the power supply chip U2 through the fifth resistor R5 and the seventh resistor R7 connected in series, the sixth resistor R6 and the fourth capacitor C4 are connected in parallel between the first pin and the second pin of the second operational amplifier U4.

In the smart back posture corrector provided above, the machine body 103 is attached to the user's back, when the user's sitting posture is incorrect, such as skewed or bent, the gyroscope will perform angular motion, and when the angular motion data exceeds the set value, the physical action part will perform physical actions to remind the user that the sitting posture is incorrect, until the user adjusts the sitting posture, in such a way that the smart back posture corrector provided by the present embodiment can accurately remind the user and judge the sitting posture of the user, so as to prevent hunchback caused by incorrect sitting posture, and the angular motion data and a data of the physical action part will be transmitted to the mobile terminal for data statistics.

The physical action part may be a vibrator. When the angular motion data monitored by the gyroscope exceeds the set value, the control circuit board controls the vibrator to turn on and the vibrator vibrates, the set value here can be a bending angle of the waist or other things.

The physical action part may be a pulser. When the angular motion data monitored by the gyroscope exceeds the set value, the control circuit board controls the pulser to turn on and the pulser sends out shock pulses.

The above embodiment uses vibration or shock pulse to act on the user's back to remind the user. Alternatively, other physical actions, such as beating, etc., may be used, depending on actual needs.

A motor is provided in the vibrator, the motor is connected with an eccentric wheel, and the eccentric wheel is driven by the motor to rotate to realize the function of vibration.

An external interface 107 communicating with the cavity is formed on the casing, and a charging port is provided on the machine body 103, when the machine body 103 is placed in the cavity, the charging port and the external interface 107 are aligned and connected with each other, in such a way that when the machine body 103 is accommodated in the casing, the machine body 103 can be charged through a data cable 200, which passes through the external interface 107 and is inserted into the charging port. During the charging process, the machine body 103 is accommodated in the casing, which will not affect the use of the user and is safe.

The casing comprises a bottom casing 106 and an upper casing 101 that swings up and down relative to the bottom casing 106, a side of the upper casing 101 is hinged with a side of the bottom casing 106, in such a way that when the upper casing 101 swings upward relative to the bottom casing 106, the cavity of the casing is in an open state, which is convenient for putting the machine body 103 into the cavity or removing the machine body 103 from the cavity of the casing; when the upper casing 101 swings downward relative to the bottom casing 106, the upper casing 101 is covered on the bottom casing 106, at this time, the upper casing 101 and the bottom casing 106 are enclosed to form the cavity, and the cavity is in a closed state.

A bottom frame 104 is provided in the cavity, the bottom frame 104 is arranged on the bottom casing 106, there is a bottom space for accommodating the adhesive sheet 302 between the bottom frame 104 and the bottom casing 106, and the bottom frame 104 is provided with a lower recess for placing the machine body 103. The lower recess is provided to facilitate the positioning of the machine body 103 in the cavity, and the bottom space is provided to facilitate the accommodation of the adhesive sheet 302.

The cavity of the casing is provided with an alcohol cotton 301, which can be stored in the bottom space. When the machine body 103 is needed for use, the alcohol cotton 301 is used to disinfect the machine body 103. Or the alcohol cotton 301 can also be stored in other locations of the cavity depending on the actual situation.

The bottom frame 104 comprises a frame plate, an upper of the frame plate forms the lower recess, a bottom of the frame plate extends downward with a bottom brace 105, the bottom brace 105 is connected to the bottom casing 106 by magnetic attraction, and the bottom space is formed between the frame plate and the bottom casing 106. The magnetic attraction method facilitates the assembly and arrangement of the bottom frame 104.

The upper casing 101 has an inner surface facing the bottom casing 106, a positioning plate 102 is connected to the inner surface of the upper casing 101, the positioning plate 102 is provided with a plurality of positioning holes, and when the upper casing 101 is covered on the bottom casing 106, an upper of the machine body 103 is penetrated between a plurality of positioning holes. In this way, when the machine body 103 is placed in the cavity of the casing, the machine body 103 can be firmly placed in the cavity of the casing under the restriction of the lower recess and a plurality of positioning holes, so as to avoid the dislocation movement of the machine body 103 in the cavity of the casing due to shaking.

A side wall of each positioning hole extends toward the inner surface of the upper casing 101 to form a positioning ring piece, and the positioning ring piece abuts against an outer circumference of the machine body 103. The positioning ring piece surrounds the outer circumference of the machine body 103 and abuts against the outer circumference of the machine body 103, which has the effect of restricting and positioning the machine body 103. The positioning ring piece is provided to increase an abutted area of the positioning plate 102 against the machine body 103, thereby enhancing the positioning function of the positioning plate 102.

The above descriptions are only the preferred embodiments of the present disclosure and are not intended to limit the present disclosure, any modification, equivalent substitution and improvement made within the principle of the present disclosure shall be included in the protection scope of the present disclosure.

What is claimed is:

1. A smart back posture corrector, comprising a machine body for attaching to a user's back and a casing, wherein the machine body and an adhesive sheet for attaching the machine body to the user's back are provided in the casing,
a control circuit board, a gyroscope and a physical action part are provided in the machine body, when an angular motion data monitored by the gyroscope exceeds a set value, the control circuit board controls the physical action part to turn on, and the physical action part outputs physical actions acting on the user's back.

2. The smart back posture corrector of claim 1, wherein a circuit of the control circuit board comprises
a control chip U1, a power supply chip U2, a first operational amplifier U3, a second operational amplifier U4, a first capacitor C1, a second capacitor C2, a third capacitor C3, a fourth capacitor C4, a fifth capacitor C5, a sixth capacitor C6, a first resistor R1, a second resistor R2, a third resistor R3, a fourth resistor R4, a fifth resistor R5, a sixth resistor R6, a seventh resistor R7, an eighth resistor R8, a ninth resistor R9 and an external power supply VCC,
an X-axis output port Xout, a Y-axis output port Yout and a Z-axis output port Zout of the control chip U1 are grounded through the first capacitor C1, the second capacitor C2 and the third capacitor C3 respectively,
a sleep control port Sleep of the control chip U 1 is connected to an operating voltage VDD through the first resistor R1, a mode selection port g_Select and a self test function port Self Test of the control chip U1 are directly grounded.

3. The smart back posture corrector of claim 2, wherein when the angular motion data exceeds the set value, the X-axis output port Xout, the Y-axis output port Yout and the Z-axis output port Zout of the control chip U1 output voltages in three directions of the X-axis, Y-axis and Z-axis respectively, in such a way that the physical action part is turned on.

4. The smart back posture corrector of claim 1, wherein the control circuit board comprises a wireless communication component for communicating with an external mobile terminal, and the mobile terminal is used for controlling the working state of the smart back posture corrector.

5. The smart back posture corrector of claim 1, wherein the physical action part is a vibrator.

6. The smart back posture corrector of claim 1, wherein the physical action part is a pulser.

7. The smart back posture corrector of claim 1, wherein a cavity for placing the machine body is provided in the casing, an external interface communicating with the cavity is formed on the casing, and a charging port is provided on the machine body, when the machine body is placed in the cavity, the charging port and the external interface are aligned and connected with each other.

8. The smart back posture corrector of claim 7, wherein the casing comprises a bottom casing and an upper casing that swings up and down relative to the bottom casing, a side of the upper casing is hinged with a side of the bottom casing, and the upper casing and the bottom casing are enclosed to form the cavity.

9. The smart back posture corrector of claim 8, wherein a bottom frame is provided in the cavity, the bottom frame is arranged on the bottom casing, a bottom space for accommodating the adhesive sheet is provided between the bottom frame and the bottom casing, and the bottom frame is provided with a lower recess for placing the machine body.

10. The smart back posture corrector of claim 9, wherein the bottom frame comprises a frame plate, an upper of the frame plate forms the lower recess, a bottom of the frame plate extends downward with a bottom brace, the bottom brace is connected to the bottom casing by magnetic attraction, and the bottom space is formed between the frame plate and the bottom casing.

11. The smart back posture corrector of claim 8, wherein the upper casing has an inner surface facing the bottom casing, a positioning plate is connected to the inner surface of the upper casing, the positioning plate is provided with a plurality of positioning holes, and when the upper casing is covered on the bottom casing, an upper of the machine body is penetrated between a plurality of positioning holes.

12. The smart back posture corrector of claim 11, wherein a side wall of each positioning hole extends toward the inner surface of the upper casing to form a positioning ring piece, and the positioning ring piece abuts against an outer circumference of the machine body.

13. The smart back posture corrector of claim 1, wherein an alcohol cotton is provided in the casing.

14. The smart back posture corrector of claim 3, wherein the power supply chip U2 provides a reference voltage Vref for the first operational amplifier U3 and the second operational amplifier U4 to compare an in-phase input voltage and an inverse-phase input voltage, a reference voltage input port Vref of the power supply chip U2 is connected to an in-phase input port of the first operational amplifier U3 through the seventh resistor R7, a ground port GND of the power supply chip U2 is directly grounded, a power port VCC of the power supply chip U2 is directly connected to the external power supply VCC, and a voltage output port Vout of the power supply chip U2 is connected to an inverse-phase input port of the first operational amplifier U3 through the eighth resistor R8, and the sixth capacitor C6 is connected between the ground port GND and the power port VCC of the power supply chip U2.

15. The smart back posture corrector of claim 14, wherein an output port of the first operational amplifier U3 is connected to a physical action part driving circuit, which is used to drive the physical action part to output the physical actions acting on the user's back, the ninth resistor R9 is connected between the output port and the inverse-phase input port of the first operational amplifier U3, a ground port of the first operational amplifier U3 is directly grounded, a power port of the first operational amplifier U3 is directly connected to the external power supply VCC, and the power port of the first operational amplifier U3 is grounded through the fifth capacitor C5.

16. The smart back posture corrector of claim 15, wherein an output port of the second operational amplifier U4 is connected to a gyroscope acquisition circuit, which is used to acquire the angular motion data of the machine body, an inverse-phase input port of the second operational amplifier U4 is connected to the Z-axis output port Zout of the control chip U1 through the second resistor R2, an in-phase input port of the second operational amplifier U4 is grounded through the third resistor R3, is connected to the external power supply VCC through the fourth resistor R4, and is connected to the reference voltage input port Vref of the power supply chip U2 through the fifth resistor R5 and the seventh resistor R7 connected in series, the sixth resistor R6 and the fourth capacitor C4 are connected in parallel between the output port and the inverse-phase input port of the second operational amplifier U4.
